(19) United States
(12) Patent Application Publication
(10) Pub. No.: US 2026/0224208 A1
(43) Pub. Date: Aug. 6, 2026

(54) SURGICAL DEVICE

(71) Applicant: ZURIMED TECHNOLOGIES AG, Zurich (CH)

(72) Inventors: Elias BACHMANN, Zürich (CH); Pol BANZET, Zürich (CH); Xiang LI, Uster (CH)

(73) Assignee: ZURIMED TECHNOLOGIES AG, Zurich (CH)

(21) Appl. No.: 19/153,173

(22) PCT Filed: Mar. 6, 2024
(86) PCT No.: PCT/EP2024/055924

§ 371 Date: Aug. 1, 2025

(30) Foreign Application Priority Data
Mar. 7, 2023 (CH) CH0002472023

(51) Int. Cl. A61B 17/00

(52) U.S. Cl. CPC A61B 17/00234; A61B 2017/00238; A61B 2017/00349; A61B 2017/00477

(57) ABSTRACT

Disclosed is a surgical device (1) is for attaching a non-woven textile to human or animal soft tissue by releasing sections of individual fibers of the non-woven textile and carrying them into the human or animal soft tissue, the device comprising a carrier (4) extending in a longitudinal direction (x), comprising a guide rod (5) and a felting needle (6) attached to a distal end of the guide rod (5), wherein the guide rod (5) comprises a dorsal end (13) which is coupled to a driving mechanism (14) via a force limiting element (15) which during operation limits the force applied by the driving mechanism (14) to the guide rod (5) to a predetermined force threshold when the working tip (7) is moved from a retracted position to an extended position.

### FIELD OF THE INVENTION

The present disclosure relates to a surgical device for attaching a non-woven textile to human or animal soft tissue.

### BACKGROUND OF THE INVENTION

Implantable non-woven textiles, in particular felts, are used in various applications. For example, they are used for general, capsular and cardiac surgery. Traditionally the felts were connected to tissue using conventional suturing techniques. A disadvantage of achieving a connection using suturing is that the quality of the connection between the felt and the tissue is limited by the number of stitches and that, should the felt experience high force, the threads can pull through the tissue resulting in so-called “cheese wiring” which causes damage to the tissue.

Therefore, surgical devices were developed in which a felting needle repeatedly pushes through the felt and into the tissue, thereby depositing, into the tissue, sections of fiber of the felt material. By moving the needle across the felting material, a strong and well distributed connection between the felt and the tissue is created.

WO2022100833A1 discloses a surgical device with a felting needle configured to move reciprocally. The surgical device includes a guide tube and a translational rod disposed within the guide connected to the needle. Disclosed are needle protection mechanisms which protect the needle from being damaged during reciprocal motion due to a contact with a rigid structure. The needle protection mechanisms are arranged between the translational rod and the needle.

### SUMMARY OF THE INVENTION

It is an object of the present disclosure to provide a surgical device. In particular, it is an object of the present disclosure to provide a surgical device for attaching a non-woven textile to human or animal soft tissue which does not have at least some of the disadvantages of the prior art.

Disclosed is a surgical device. The surgical device is suitable for attaching a nonwoven textile to human or animal soft tissue by releasing sections of individual fibers of the non-woven textile and carrying them into the human or animal soft tissue. The sections of individual fibers may be pushed, dragged, or otherwise brought or deposited into the human or animal soft tissue, where they remain, thereby attaching, connecting, or anchoring the non-woven textile into the soft tissue. The surgical device comprises a carrier extending in a longitudinal direction, the carrier comprising a guide rod and a felting needle, wherein the felting needle is attached to a distal end of the guide rod. The felting needle is an elongated member including a working tip at a distal end, the working tip preferably comprising, for example, a sharp tip, a blade, a blunt, or a flat end or a combination thereof. The guide rod and the felting needle are at least partially arranged in a guide bushing and the felting needle is reciprocally movable between an extended position, in which the working tip of the felting needle protrudes beyond an aperture arranged at a distal end of the guide bushing, and a retracted position, in which the working tip of the felting needle does not protrude beyond the aperture arranged at the distal end of the guide bushing. The guide rod is coupled, at a distal end, to a driving mechanism via a force limiting element which, during operation, limits the force applied by the driving mechanism to the guide rod to a predetermined force threshold when the working tip is moved from the retracted position to the extended position.

In an embodiment, the force limiting element is further configured to not limit the force applied by the driving mechanism to the guide rod to the predetermined force threshold when the working tip is moved from the extended position to the retracted position. Thereby, the transfer of force during retraction of the working tip is not limited to the predetermined force threshold. In particular, the driving mechanism may apply a force greater than the predetermined force threshold to the guide rod, when the working tip is retracted. This is beneficial for moving the working tip back to the retracted position in a situation where the working tip may have become lodged or partially stuck, in particular where the force limiting element may have engaged during movement of the working tip from the retracted position to the retracted position.

In an embodiment, the force limiting element is configured to have a predetermined retraction force threshold which during operation limits the force applied by the driving mechanism to the guide rod to a predetermined retraction force threshold when the working tip is moved from the extended position to the extended position. The predetermined retraction force threshold may be different than the predetermined force threshold during extension, in particular, in that the predetermined retraction force threshold may be greater than or less than the predetermined force threshold during extension.

According to an implementation, the force limiting element comprises a deformable element which deforms when the predetermined force threshold is reached in longitudinal direction, thereby interrupting the force transmission in longitudinal direction. Preferably, the force limiting element does not attenuate the force applied by the driving mechanism until the predetermined force threshold is met. Preferably, the force limiting element is configured to return to an initial state when the applied force is removed and/or the felting needle is returned to the retracted position. Preferably, the force limiting member does not break when the predetermined force threshold is exceeded.

For example, the deformable element is a buckling element which extends between a first end and a second end in the longitudinal direction and deforms in a lateral direction, the lateral direction being orthogonal to the longitudinal direction, when the predetermined force threshold is reached. The buckling element is configured such that it buckles when the predetermined force threshold is reached. The buckling element may have a round, rectangular, oval and/or V-shaped cross-section.

The force limiting mechanism may exhibit hysteresis. For example, the hysteresis may be exhibited in the displacement of the working tip of the felting needle in the longitudinal direction as a function of the displacement of the driving mechanism in the longitudinal direction in situations where the force on the working tip in the longitudinal direction (in particular a force directed towards the dorsal end of the working tip) exceeds the predetermined force threshold. In particular, the displacement of the working tip of the felting needle from a retracted position to an extended position, as a function of the displacement of the driving mechanism, may be different than the displacement from the extended position back to the retracted position. Additionally or alternatively, the hysteresis may be exhibited in the force exerted on the working tip of the needle in the longitudinal direction as a function of the displacement of the driving mechanism in the longitudinal direction in situations where the force on the working tip in the longitudinal direction (in particular a force directed towards the dorsal end of the working tip) exceeds the predetermined force threshold. Specifically, during extension, if the needle hits a hard object, the force exerted on the needle by the driving mechanism ramps up rapidly until the predetermined force threshold is met, after which the force either remains constant or drops (preferably is interrupted in that the force drops sharply). For example, the driving mechanism may become substantially disengaged from the working tip. However, in opposite direction, in particular when the working tip moves from an extended position towards the retracted position, the force limiting mechanism may not limit the pulling force exerted on the working tip by the driving mechanism to the predetermined force threshold. As a result, if the working tip becomes lodged in the hard object (e.g. hard tissue) and the force threshold is reached, then the working tip can be pulled out of the hard object at a greater force, ensuring that even if carrying means engage with the hard object, the working tip can still be removed. The buckling element may be clamped at the first end and/or the second end. In particular, the buckling element may be clamped to the guide rod at the first (distal) end and/or clamped to the driving element at the second (dorsal) end. Alternatively, the buckling element may be pivotably attached at the first end and/or the second end.

In an example, the buckling element may be preformed such that the buckling element buckles when the pre-determined force threshold is reached during operation. The preforming may include stressing the buckling element, annealing the buckling element, and/or bending the buckling element. The preforming may include introducing imperfections such as indentations, defects, stress risers, etc.

In an example, the working tip of the felting needle includes carrying means for supporting the carrying of sections of individual fibers of the non-woven textile into the soft tissue.

In an example, the carrying means include an essentially flat end section arranged at a distal end of the working tip. The essentially or substantially flat end section may include one or more flat and/or curved surfaces arranged substantially orthogonal to the longitudinal direction. The flat end section may have, for example, a round, oval, and/or rectangular cross-section. The flat end section may include protrusions and/or recesses designed to catch, snag, and/or pull on fibers. The flat end section may have a surface roughness.

Additionally or alternatively, the carrying means of the working tip may include protrusions and/or recesses along an outer shaft of the working tip for carrying sections of the individual fibers into the soft tissue. The protrusions and/or recesses may be designed as indentations or recesses into the outer shaft, and/or as protrusions from the outer shaft. The protrusions and/or recesses may include, for example, hook elements, a crown, a notch, and/or a barb. The working tip may have a circular cross-section, a triangular cross section, a star-shaped cross-section, a helical cross section and/or a screw thread cross section.

According to an implementation, the surgical device comprises a frame to which the guide bushing and the driving mechanism are attached.

The frame may comprise a recess, in which the force limiting element is arranged between the dorsal end of the guide bushing and the driving mechanism. The recess may be designed to accommodate a buckling of the buckling element, in particular in that it has a lateral dimension larger in extent than an extent of the buckling element.

In an implementation, a channel extends in longitudinal direction between the guide rod and the guide bushing. The channel prevents a buildup of pressure differences in the longitudinal direction, thereby preventing a pumping effect from occurring in which the reciprocal motion causes a fluid, for example air, to be pumped in the distal direction. The channel preferably extends over half of the distance between the guide rod and the guide bushing.

For example, the guide rod comprises or defines a channel which extends in longitudinal direction. Additionally or alternatively, the guide bushing comprises or defines a channel.

The driving mechanism may comprise an adapter to connect the driving mechanism to an external actuator. The external actuator is configured to drive the driving mechanism via the adapter, by engaging with the adapter. The external actuator may be releasably connected to the surgical device, for example using a releasable snap-fit mechanism. The external actuator may be powered by electrical energy, pneumatic energy, etc.

In an embodiment, the aperture of the guide bushing is formed by a guide bushing collar which has an inner diameter less than an inner diameter of the guide bushing. The guide bushing collar includes a transitional surface at a distal end and/or at a dorsal end. The transitional surface(s) are designed to ensure that the felting needle extends and retracts in and out of the guide bushing collar correctly, in particular that the working tip of the felting needle does not snag or catch on the guide bushing collar even if a lateral force is applied to the felting needle during extension or retraction. The transitional surfaces may include one or more bevels, chamfers, and/or fillets, etc. Essentially, the transitional surfaces are oriented away from the longitudinal direction such that the felting needle, if incident on the transitional surfaces, is deflected towards the longitudinal direction.

In a preferred variation, the felting needle is made from a metal, for example a metal alloy such as stainless steel or nitinol. The guide rod is made, for example, from an injection molded plastic material which encompasses the felting needle opposite to the working tip at least partially, thereby securely attaching the felting needle to the guide rod. The frame may be made from an injection molded fiber reinforced plastic material.

In an implementation, the force limiting element and the felting needle are integrally formed and extend through the guide rod.

In an implementation, the guide rod comprises at least two opposite indentations between which a part of the force limiting member is arranged.

In a variation, the force limiting element is coupled to the driving mechanism by a piston rod. The driving mechanism may also include the piston rod. The piston rod may be connected to a sliding pin attached to a rotating element driven by the external actuator.

In a variation, the force limiting element is coupled to the driving mechanism by a scotch yoke. The scotch yoke includes a sliding yoke and a connecting rod, the connecting rod attached to the force limiting element. The scotch yoke further includes a sliding pin attached to the rotating element driven by the external actuator.

The rotating element may be a crank, a wheel, or a disc, for example.

The frame may include a cylinder arranged in the longitudinal direction. The driving mechanism may include a piston (or a connecting rod) arranged at least partially inside the cylinder. The piston is connected at a distal end to the force limiting element and at a dorsal end to the piston rod. The cylinder is configured to guide the piston or connecting rod through reciprocal motion when the surgical device is in operation.

The frame may be at least partially encompassed by an outer housing. The housing may comprise two housing halves made of injection molded plastic material.

The guide rod may comprise at least two guide surfaces spaced apart from each other in longitudinal direction. The guide surfaces are preferably designed to provide a low friction interface between the guide rod and the guide bushing.

It is to be understood that both the foregoing general description and the following detailed description present embodiments of the disclosure, and are intended to provide an overview or framework for understanding the nature and character of the disclosure. The accompanying drawings are included to provide a further understanding, and are incorporated into and constitute a part of this specification.

The drawings illustrate various embodiments, and together with the description serve to explain the principles and operation of the concepts disclosed.

### BRIEF DESCRIPTION OF THE DRAWINGS

The herein described invention will be more fully understood from the detailed description given herein below and the accompanying drawings which should not be considered limiting to the invention described in the appended claims. The drawings are showing:

FIG. 1 shows schematically a side view of a surgical device;

FIG. 2 shows schematically an exploded perspective view of a surgical device;

FIG. 3 shows schematically a perspective view of a carrier;

FIG. 4 shows schematically a perspective view of a section of a carrier including a distal end of a guide rod and a felting needle;

FIG. 5 shows schematically a perspective view of a working tip of a felting needle;

FIG. 6 shows schematically a perspective view, including interior lines, of a section of a carrier including a dorsal end of a guide rod and part of a force liming element;

FIG. 7 shows schematically a cut-away perspective view of a surgical device;

FIG. 8 shows schematically a cut-away perspective view of a distal end of a guide bushing, further showing a distal end of a carrier including part of a guide rod and a felting needle in a retracted position;

FIG. 9 shows schematically a cut-away perspective section view of part of a surgical device including in particular a force liming element and a driving mechanism;

FIG. 10 shows schematically three plan views of a surgical device in which the felting needle is in a retracted position, in an extended position, and in which the felting needle is partially extended and in contact with a surface such that the force liming element is engaged; and

FIG. 11 shows two charts illustrating the needle displacement and force exerted on the needle tip as a function of the driving mechanism displacement; and

### DESCRIPTION OF THE EMBODIMENTS

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

FIG. 1 shows a side on view of a schema of a surgical device 1. The surgical device 1 is a hand held device which is typically operated by a surgeon. The surgical device 1 is used to connect a non-woven textile, such as a felt material, to soft tissue in a human or animal body. The surgical device 1 is a sterilized device which is typically manufactured and assembled in a clean room environment. The surgical device 1 is typically sterilized after assembly and stored in a sterile package for use in a surgery. After use, the surgical device 1 must either be cleaned and sterilized, or discarded.

For sterilization, for example during or after manufacture, or after use, it is important that the distal end of the surgical device 1, in particular any part which enters or comes into contact with the human or animal, is readily accessible by sterilization fluids. In particular, the sterilization fluid must be able to circulate freely across all exterior and interior surfaces of the surgical device 1.

The surgical device 1 has an outer housing 28 made of a plastic material formed by injection molding. The outer housing 28 defines a grip area where the surgical device 1 may be held by the surgeon. The outer housing 28 further defines an upper part which is elongated in the longitudinal direction, the upper part including an opening designed to accept a guide bushing 28. The outer housing 28 is formed from two parts which are attached to each other along a join line. The join line extends around the outer housing 28 substantially in a lateral (y-z) plane. Thereby, a risk of pinching of surgical gloves in the join line during operation is reduced. The two parts are joined using, for example, a press-fit connection, bonding, and/or additional retaining mechanisms including a snap fit mechanism, screws, etc.

The guide bushing 8 is attached to the outer housing 28, in particular the opening of the outer housing 28, at a dorsal end 28 of the guide bushing 8. The guide bushing 8 has an elongated shape, for example a hollow cylindrical shape, for example a hollow circular cylindrical shape, with a diameter of 2 mm to 10 mm, preferably 3 mm to 8 mm, more preferably 5 mm. The guide bushing 8 has a longitudinal extension of 2 cm to 30 cm, preferably 5 to 20 cm, more preferably 15 cm. The guide bushing 8 has a distal end 19 which includes an aperture 11. The distal end 19 may be beveled, chamfered, and/or filleted. The guide bushing 28, at the dorsal end, is open to an interior of the outer housing 28.

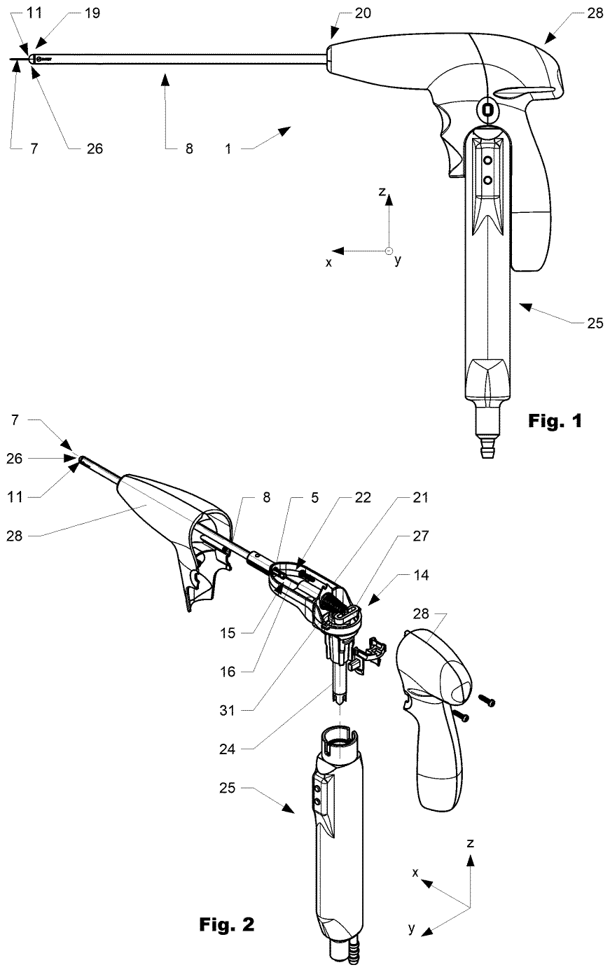

The guide bushing 8 at least partially encloses a carrier 4 (not shown), which carrier 4 comprises a guide rod 5 and a felting needle 6. The guide rod 5 has a cross sectional shape which is at least partially complementary to an interior cross section of the guide rode 5, such that the guide rod 5 may move reciprocally (i.e. back and forth) inside the guide bushing 8 in a longitudinal direction without excessive lateral motion. The carrier 4 is explained in more detail below with reference to FIGS. 2 to 4.

In an embodiment, the guide bushing 8 may be flexible at least over part of its length. The guide bushing 8 may be implemented as a form of catheter. The guide bushing 8 may be flexible over its entire length. The guide bushing 8 may be bent into a particular shape, and retain that shape after bending. The guide bushing 8 may therefore be formed into a specific shape for a particular surgery, for example to reach a surgery site in an optimal way. Flexible as mentioned herein may be understood in that the guide bushing 8 may be bent, in particular while navigating a twisting path of a hollow organ (e.g. a blood vessel) and/or is capable of being brought into a bent shape. Preferably the guide bushing 8 is flexible over its entire length. Alternatively, the guide bushing 8 may have one or more flexible section(s).

A working tip 7 of the felting needle 6 is shown extending out of (i.e. beyond) the aperture 11 in an extended position. In a retracted position, the working tip 7 of the felting needle 6 is retracted inside the aperture 11.

An external actuator 25 is releasably connected to the surgical device 1 and drives the driving mechanism (not shown). The external actuator 25 may be connected using a snap fit connector, for example a cantilever snap fit connector. A part of the cantilever arm extends through the outer housing 28 which, when depressed, causes the snap fit connection to disengage and release the external actuator 25. When the external actuator 25 is turned on, the working tip 7 of the felting needle 6 moves back and forth in reciprocal motion between the retracted position and the extended position. Preferably, the frequency of reciprocal motion is between 1-200 Hz, preferably 10-100 Hz, most preferably 20-80 Hz and in a particular example, 40 Hz. When the external actuator 25 is turned off, the working tip 7 returns to the retracted position as is explained below in more detail with reference to FIG. 2.

FIG. 2 shows an interior of the surgical device 1. A frame 21 is arranged inside the outer housing 28. The frame 21 has, at a distal end in the longitudinal x direction, an opening for receiving a dorsal end of the carrier 4, in particular the guide rod 5. The frame 21 has, adjacent to the opening, a recess 22 within which the force liming element 16 is arranged. The recess 22 is designed such that the force limiting element 16, in particular a buckling element 16, has sufficient space to buckle in a lateral x y direction. The buckling element 16 is preferably configured to buckle in the lateral x y direction when a predefined force threshold is met. The force liming element 16 is connected to the carrier 4, in particular to the guide rod 5. For example, the force liming element 16 is clamped to a dorsal end of the guide rod 5.

The force liming element 16 is connected, at a dorsal end 17, to a driving mechanism 14. The driving mechanism 14 is connected, during operation, to the external actuator 25. The driving mechanism 14 may include an adapter 24 configured to engage with a rotating end of the external actuator 25. The driving mechanism 14, in particular the adapter 24, is configured to translate a rotational motion of the external actuator to a reciprocating motion for driving the carrier 4 in a reciprocating motion. The driving mechanism 14 may include a piston rod, a scotch-yoke 27, or another suitable mechanism translating the rotational motion. Specifically, the external actuator 25 is configured to rotate in the x y plane. The adapter 24 engages with the external actuator 25.

The adapter 24 may include a sliding pin which rotates in a horizontal x-y plane. The sliding pin may be arranged on a crank, wheel, or disc, for example, of the adapter 24. Specifically, the sliding pin revolves, in a horizontal x y plane, about an axis of rotation which extends in the z direction.

In an example, the force liming element 16 is directly connected to the sliding pin. For example, the force liming element 16 may comprise a ring part mounted on the sliding pin such that the force liming element 16 is directly driven by the circular motion of the sliding pin, the force liming element 16 thereby also acting as a piston rod.

Alternatively, the sliding pin engages with a piston rod or a yoke of a scotch yoke connector.

The adapter 24 is configured such that a diameter of rotation of the sliding pin is between 5-40 mm, preferably 10-30 mm, most preferably 15-20 mm. The diameter of rotation defines a range of motion of the carrier 4. Specifically, the diameter of rotation defines a range of motion of the working tip 7 of the needle between a retracted position and an extended position. A maximal extension of the working tip of the needle 7 beyond the aperture 11 is defined by the diameter of rotation and a distance between the aperture 11 and the working tip 7 when the working tip 7 is in a retracted position. Preferably, the surgical device 1, in particular the diameter of rotation and a distance between the aperture 11 and the retracted working tip 7, is designed such that the maximal extension of the working tip 7 is between 1-30 mm, preferably 6-15 mm. Particular examples of the maximal extension are 6.5 mm, 8.5 mm, or 12 mm.

The working tip 7 includes carrying means configured to carry (e.g., transport by pushing, pulling, or dragging) sections, i.e. lengths, of fibers of the non-woven textile into the soft tissue as the working tip 7 moves through the non-woven textile into the soft tissue.

The carrying means may be arranged on a distal end of the working tip 7. The carrying means thereby advantageously enable the working tip 7 to deposit the lengths of fibers at the maximum penetration depth of the working tip 7. Carrying means arranged on the distal end of the working tip 7 include, for example, a flat end section 41 of the working tip 7.

Additionally or alternatively, the carrying means may be arranged on an outer shaft 40 of the working tip.

The driving mechanism 24 includes a biasing member 31, such as a coil spring, configured to return the carrier 4 to a retracted position when the external actuator is turned off. Specifically, the biasing force of the biasing member 31 is configured to exceed a breakaway force of the external actuator 25. The biasing member 31 is connected at one end to the frame 21, in particular a lateral interior wall of the frame 21, and at another end to the adapter 24. The lateral interior wall is arranged in the y-z plane and divides the frame 21 into a distal section which substantially encloses the force liming element 16, and a dorsal section in which the driving mechanism 14 is substantially arranged. The biasing member may be arranged around a dorsal end of the force liming element 16 to achieve a compact design.

The frame 21 further includes a bore 32 oriented in the longitudinal direction x, the bore 32 configured to restrict a motion of the driving mechanism 14 and/or the force liming element 16 to the longitudinal direction. More specifically, a piston or a connecting rod of the driving mechanism 14 and/or the force liming element 16 is arranged in the bore 32. The bore 32 is preferably arranged adjacent to the interior wall of the frame 21.

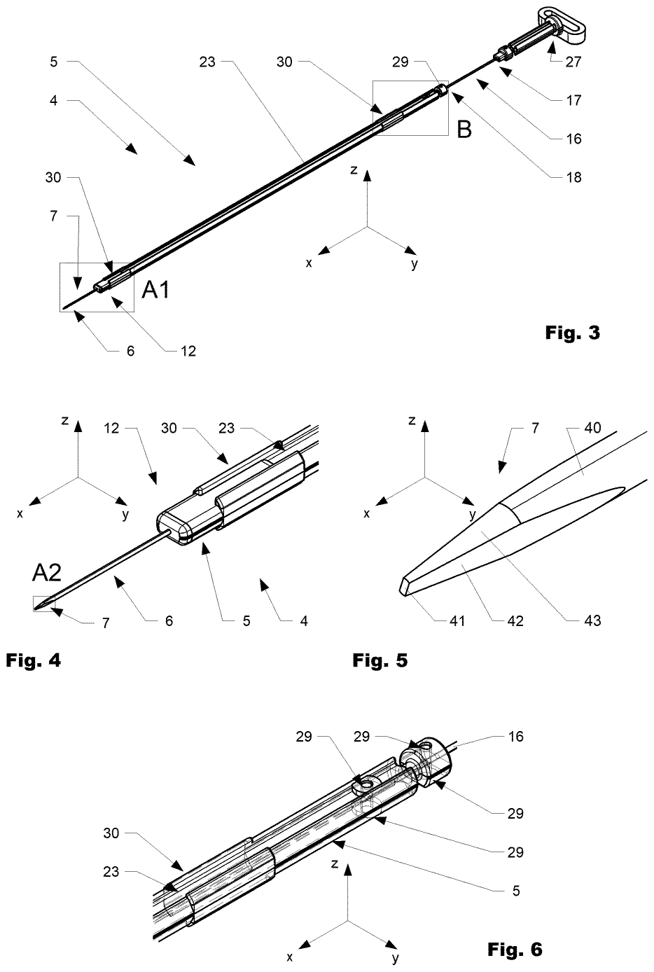

FIG. 3 shows schematically a perspective view of a carrier 4. The carrier 4 comprises a felting needle 6 and a guide rod 5. The felting needle 6 is arranged at a distal end (in the positive longitudinal direction x) of the carrier 4 and is connected to the guide rod 5. The carrier 4 is connected at a dorsal end to a force liming element 16. The guide rod 5 has a cylindrical shape, for example a circular, oval, or rectangular cylindrical shape with a cross sectional extension (e.g., a diameter) of 2 mm to 8 mm, preferably 5 mm. The guide rod 5 is preferably made of injection molded plastic material. The guide rode 5 may have edge fillets.

The carrier 4 is manufactured, for example, by arranging a dorsal section of the felting needle 6 and a distal section of the force liming element 16 into a mold and, and then injection molding plastic material to form the guide rod 5.

In an embodiment, the felting needle 6 and the force liming element 16 are integrally formed, i.e. are made of one continuous part, and the guide rod 5 is formed by injection molding plastic material around the felting needle 6 and the force liming element 16.

The guide rod 5 may include one or more channels 23 on an exterior surface, which channels 23 extend at least along part of the exterior surface of the guide rod 5. The channel(s) 23 serve to prevent a buildup of pressure inside the guide bushing 8 due to the reciprocal motion of the carrier 4 when the surgical device 1 is in operation.

The channel(s) 23 may further engage with guiding means on an interior surface of the guide bushing 8, which guiding means have a complementary shape to the guide bushing 8 and serve to guide the guide rod 5 within the guide bushing 8 in a pre-determined orientation (i.e., at a pre-determined angle in the y z plane). In other words, the channel(s) 23 may engage with the guiding means to prevent a rotation of the guide ride 8 about the longitudinal axis x.

The guide rod 5 may further include one or more guide surfaces 30 configured to slidably engage with the guide bushing 8. The guide surfaces 30 may be formed of a different material than the remainder of the guide rod 5. For example, the guide surfaces 30 may be formed of a material which reduces friction between the guide rod 5 and the guide bushing 8, such as PTFE. The guide surfaces 30 may have a lateral cross section that is at least partially complementary to a lateral interior cross section of the guide bushing 8. The guide surfaces 30 have a defined longitudinal extension, for example a longitudinal extension of approximately 10 mm. The guide surfaces 30 are preferably arranged at or near a distal end of the guide rod 5 and at or near a dorsal end of the guide rod 5, i.e. within 3 cm of the dorsal and/or the distal end of the guide rod.

The guide surfaces 30 preferably do not surround an entire section of the guide rod 8, preferably providing for at least one gap to allow for air to move past the guide surfaces 30 to prevent any pressure buildup.

In an embodiment, the guide rod 5 is flexible over at least part of its length. The guide rod 5 may be flexible over its entire length. The guide rod 5 may also be flexible only in particular sections. The guide rod 5 may be implemented as a flexible driving element configured to transmit the driving force from the driving mechanism 14 to the felting needle 6. The flexible driving element may be implemented as a driving wire, for example, connected at a distal end to the felting needle 6 and at a proximal end to the force limiting element 16. Optionally, guide elements are arranged along the driving element in longitudinal direction for reducing a friction between the driving element and the guide bushing 8. The guiding elements may eliminate contact between the driving element and the guide bushing 8. The guiding elements may be implemented as concentric lead elements which are circular, ring-shaped and extend from an inner side of the catheter. The guiding elements preferably also have a low friction index to allow optimal guidance and force transmission. The guiding elements reduce bending forces and ultimately reduce vibrations during application. The guiding elements center the driving wire in the guide bushing 8, thereby preventing a bending or buckling of the driving wire in the guide bushing 8 beyond the bending of the guide bushing 8. The guiding elements are preferably covered by a PTFE coating to lower a friction between the driving wire and the guiding elements.

The force liming element 16 has a distal end 18 and a dorsal end 17. The distal end 18 is connected to the guide rod 5 and the dorsal end 17 is connected to the driving mechanism 24. Optionally, the dorsal end 17 is connected to the driving mechanism 24 by way of a piston 27. The force limiting element 16 may be clamped at the distal end 18 to the guide rod 5 and/or may be clamped at the dorsal end to the piston 27.

The force liming element 16 may include a deformable element. The deformable element may be deformable by bending and/or buckling. The deformable element may be implemented as a buckling element 16, more particularly a buckling wire. The buckling element 16 is elongated in the longitudinal x direction and is configured to buckle when the force exerted by the driving mechanism 24 on the carrier 4 (or vice versa) in a longitudinal direction x exceeds a predetermined force threshold. When the exerted force exceeds the predetermined force threshold, the buckling element 16 buckles (i.e. suddenly laterally deflects in the y-z direction) resulting in a sudden interruption of the force exerted on the carrier 4.

The buckling element 16 may have a circular, oval, rectangular, and/or substantially linear cross-section. The buckling element 16 may be preformed.

The buckling element 16 is configured to return to its original shape (i.e. linear shape) when the force no longer exceeds the predetermined threshold force and/or the working tip 7 has returned to a retracted position. Preferably, the buckling element 16 may buckle multiple times without being damaged, breaking, or otherwise being rendered inoperable. This allows for continued operation of the surgical device 1 even after the predetermined force threshold has been exceeded.

The buckling element 16 is further configured to transmit the translational force exerted by the driving mechanism without substantial attenuation (e.g., without any elastic deformation or damping) prior to the predetermined force threshold being met. This ensures a direct transmission of force during normal operation.

The buckling element 16 is preferably configured to buckle in the y direction. To this end, the buckling element 16 may have a cross sectional shape with greater extension in the z direction than in the y direction. For example, a rectangular cross sectional shape elongated in the z direction.

The buckling element 16 is preferably a metal, such as nitinol (a nickel and titanium alloy).

The predetermined force threshold may be due to the material type, the shape (i.e. cross-section and longitudinal extension), the fixation method, and/or any processing or working of the force liming element 16 (in particular the buckling element). The processing or working may include bending, preforming, prestressing, annealing, etc.

The force liming element 16 is configured such that the predetermined force threshold is in the range of 1 N to 20 N, preferably 2 N to 10 N, more preferably 4 N to 8 N, most preferably 5 N.

The predetermined force threshold ensures that the working tip 7 is not damaged during operation through inadvertent collision with a hard tissue, such as bone, or another surgical instrument present at a surgery site, such as a scalpel. This ensures that damage to the hard tissue is prevented and also prevents the working tip 7 of the felting needle 6 from breaking, possibly resulting in the working tip 7 or part thereof being lodged in the soft tissue of the human or animal.

FIG. 4 is a close-up of section A1 of FIG. 3 and shows a distal end of the carrier 4. In particular, the felting needle 6 attached to the distal end of the guide rod 5 is visible, along with the channel 23 of the guide rod 5. The guide rod 5 has a substantially rectangular lateral cross section with edge fillets. Guide surfaces 30 near the distal end of the guide rod 5 are shown, which guide surfaces 30 however do not completely enclose the guide rod 5, leaving a gap allowing for air to move in a longitudinal direction during operation of the surgical device 1. A channel 23 in the form of an elongated recess in the guide rod surface is shown, the channel 23 terminating at a distal end near the distal end of the guide rod 5. The distal end of the channel 23 is in a section of the guide rod 5 where the guide surfaces 30 are arranged.

The felting needle 6 has a length of 5-50 mm, preferably 10-40 mm, most preferably 15-30 mm and terminates at a distal end in the working tip 7 described in more detail with reference to FIG. 5.

The felting needle 6 may have a circular, oval, rectangular, and/or other cross section. Further, the felting needle 6 may include a helical screw.

The felting needle 6 may include, on an outer surface, protrusions and/or recesses for felting, in particular designed to transport sections of fiber into the soft tissue during operation. These protrusions and/or recesses may be in the form of barbs or hooks. In case the felting needle 6 has a rectangular cross section or have a helical screw shape, protrusions and/or recesses may be arranged on edges of the felting needle 6.

FIG. 5 a close-up of section A2 of FIG. 4 shows a working tip 7 of the felting needle 6. The working tip 7 shown has a shape similar to a flat-head screwdriver and is shown as an example only. Other working tips 7 are possible, including tips in the shape of a plus, star shaped tips, etc. The working tip 7 shown has an essentially flat end section 41. The flat end section 41 is joined to an outer surface 40 of the working tip 7 by transitional lateral beveled surfaces 42, 43.

The flat end section 41 may have a rough surface configured to catch fibers. The flat end section 41 may have protrusions and/or recesses configured to catch fibers. The protrusions and/or recesses may have a width of 0.005-0.1 mm. The protrusions and/or recesses may have a depth of 0.005-0.1 mm. The protrusions and/or recesses may have a tapering shape and, in particular, they may be semicircular, V-shaped, or U-shaped.

In a preferred embodiment, the flat end section 41 at the distal end has a first width perpendicular to the longitudinal direction of the felting needle 6 of at most 1 mm. In a preferred embodiment, the first width is at most 0.5 mm. Particularly preferred, the first width is at most 0.4 mm. Most preferred, the first width is at most 0.3 mm. These dimensions are particularly suitable for catching fibers of the non-woven textile and carrying the fibers into soft tissue. As mentioned above, these fibers then become entangled with the soft tissue. If the distal end of the felting needle is too large, too many fibers are caught at the same time and pushed towards the soft tissue. Rather than catching few fibers or only a single fiber that can be drawn out of the felt alone, this simply results in pushing too many fibers and damages the soft tissue.

In a further embodiment, the first width is at least 0.02, 0.03, 0.04 or 0.05 mm. These minimal widths ensure that the fibers can be caught by the flat end section 41. If the first width is less, then the felting needle 6 may penetrate the felt without catching any or only rarely fibers, resulting in no or poor attachment. Further, if the first width is below these values, the felting needle 6 may cut the fibers in some cases.

The flat end section 41 may also have a second width that is perpendicular to the longitudinal axis of the felting needle 6 and perpendicular to the first width. The second with may be at least 0.01, 0.02, 0.03, 0.04, or 0.05 mm. Similar consideration as for the first width apply.

The surface roughness may be expressed using mean line roughness of 3.2, 2.5, 2.0, 1.5, 1.0 μm or more. Mean line may be understood as the deviation of a surface from a mean height. The roughness may be the arithmetic average value of a roughness profile determined from deviations about the center line within the evaluation length. The roughness of the distal end and in particular the roughness of the flat end section 41 may support catching and carrying individual fibers.

FIG. 6 is a close-up of section B of FIG. 3 and shows a dorsal end of the carrier 4, in particular a dorsal end of the guide rod 5. A channel 23 on the outside surface of the guide rod 5 extends until the end of the guide rod 5. Guide surfaces 30 are arranged on the outside of the guide rod 5 near the dorsal end of the guide rod 5. The guide rod 5 is connected to a distal end of the force liming element 16, which force liming element 16 extends partially into the guide rod 5 for a secure mechanical connection.

Additionally, two pairs of indentations 29 in the guide rod 5 are shown. The indentations 29 are in a part of the guide rod 5 into which the force liming element 16 extends. The indentations 29 extend from an exterior surface of the guide rod 5 to the force liming element 16. For each pair of indentations 29, a first indentation 29 extends from an opposite surface of the guide rod 5 with respect to the second indentation 29.

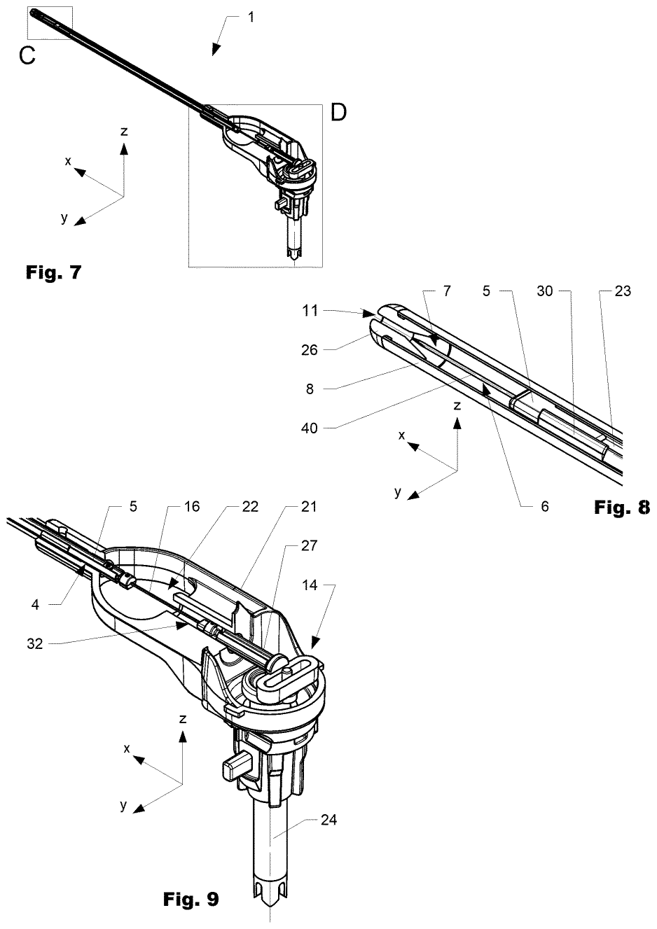

FIG. 7 shows a perspective cut away view of the surgical device 1. Sections C and D are described in more detail with reference to FIGS. 8 and 9, respectively.

FIG. 8 shows a distal end of the guide bushing 8 in which the guide rod 5 and the felting needle 6 are arranged in a retracted position. The guide bushing 8 is cylindrically shaped and encloses the guide rod 5.

The guide rod 5, in particularly the guide sections 30, are slidably engaged with the interior surface of the guide bushing 8 enabling the reciprocal motion of the felting needle 6 in the longitudinal x direction. The gap between the two guide sections 30 allows air to move past the guide rod 5. The channel 23 which terminates in an area of the guide sections 30 further allows for air movement inside the guide rod 5.

The aforementioned gaps between the guide sections 30 and the channel 23 also allow for good sterilization as they allow the sterilization fluid to easily flow through the interior of the guide bushing 8.

The guide bushing 8 includes, at a distal end, a guide bushing collar 26 which may be a separate part or integrally formed with the guide bushing 8. The guide bushing collar 26 defines an aperture 11 through which the felting needle 6 moves when the surgical device 1 is in operation. The guide bushing collar 26 is designed to guide the felting needle 6 in the longitudinal direction during reciprocal motion. The guide bushing collar 26 defines a cylinder extending in the longitudinal direction through which the felting needle 6 moves. The cylinder terminates at a distal end at the aperture 11 and has a diameter larger than a diameter of the felting needle 6.

The guide bushing collar 26 has, adjacent to the cylinder, a transitional beveled surface which is designed to guide the felting needle 6 through the cylinder and out through the aperture 11 when the felting needle 6 moves from the retracted position to the extended position.

The guide bushing collar 26 has, at a distal end, a curved outside surface.

FIG. 9 shows section D of FIG. 7 in more detail, in particular showing the frame 21, a dorsal end of the guide rod 5 of the carrier 4, and the driving mechanism 14. As shown, the driving mechanism 14 includes an adapter 24 configured to engage with the external actuator 25 (not shown). The external actuator provides rotational motion in the x-y plane. An upper part of the adapter 24, which is arranged in a dorsal section of the frame 21, includes a sliding pin mounted on a rotation wheel (a rotating disc or crank would of course is also foreseen). The sliding pin is connected to a scotch yoke which translates the rotational motion into a reciprocating motion of the piston 27, which piston 27 is connected to the force limiting element 16. Alternatively, instead of a scotch yoke, a piston rod could also be connected to the sliding pin and pivotably coupled to the piston 27. Also possible would be a direct connection between the force limiting element 16 and the sliding pin, for example by terminating the force limiting element 16, at a dorsal end, in a loop or ring which engages the sliding pin.

The recess 22, which is arranged in the distal section of the frame 21, provides space for the force limiting member 16, for example the buckling element 16, to buckle into the y direction when the force exceeds the predetermined force threshold.

The dorsal and distal sections of the frame 21 are separated by a bore 32 arranged in the longitudinal direction which is configured to receive, at least partially, the piston 27 and/or the force limiting element 16. The bore 32 guides the piston 27 and/or the force limiting element 16 in the longitudinal direction.

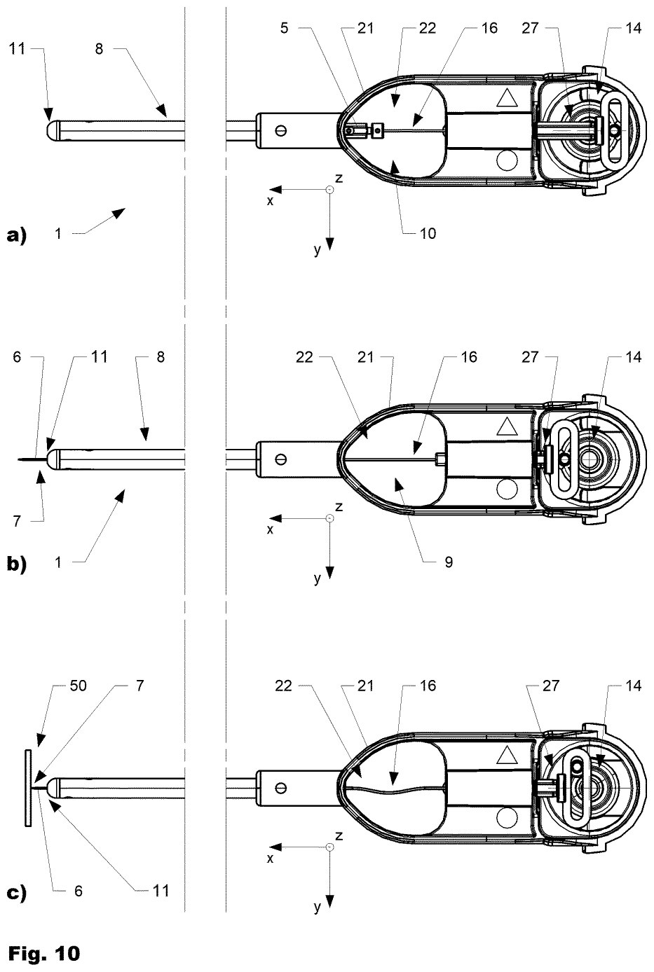

FIG. 10 shows a top down cutaway view of the surgical device 1 in three different states.

In FIG. 10 a), the carrier 4 of the surgical device 1 is in a retracted position 10 in which the working tip 7 of the felting needle 6 does not extend beyond the aperture 11. This is a default state of the surgical device 1 when it is not in operation. A biasing member (not shown) ensures that, even when the external actuator is connected, a biasing force of the biasing member returns the carrier 4 to the retracted position 10 when the external actuator is turned off. The guide rod 5 of the carrier partially enters the recess 22 in the retracted position 10, and the piston 27 extends almost completely out of the bore 32 towards the dorsal end of the surgical device 1. The sliding pin of the driving mechanism 14 is at its most dorsal position.

In FIG. 10 b), the carrier 4 of the surgical device 1 is in the extended position 9. The working tip 7 of the felting needle 6 extends beyond the aperture 11. In the extended position 9, the piston 27 almost completely enters the bore 32.

FIG. 10 c) shows a state when the working tip 7 of the felting needle 6 hits a hard object, causing the force exerted on the working tip 7 of the needle to exceed the predetermined force threshold. The force limiting element 16 buckles, in particular it deflects in the y direction, thereby causing an intermediate interruption in force transmission between the driving mechanism 14 and the felting needle 6. Thereby, damage to the felting needle 6 (in particular, the working tip 7) and/or damage to the hard object is prevented. Once the force no longer exceeds the predetermined force threshold, for example because the hard object has been removed, or the felting needle 6 has retracted, the buckling element 16 “unbuckles”, i.e. returns to its initial state. The buckling element 16 may then buckle again.

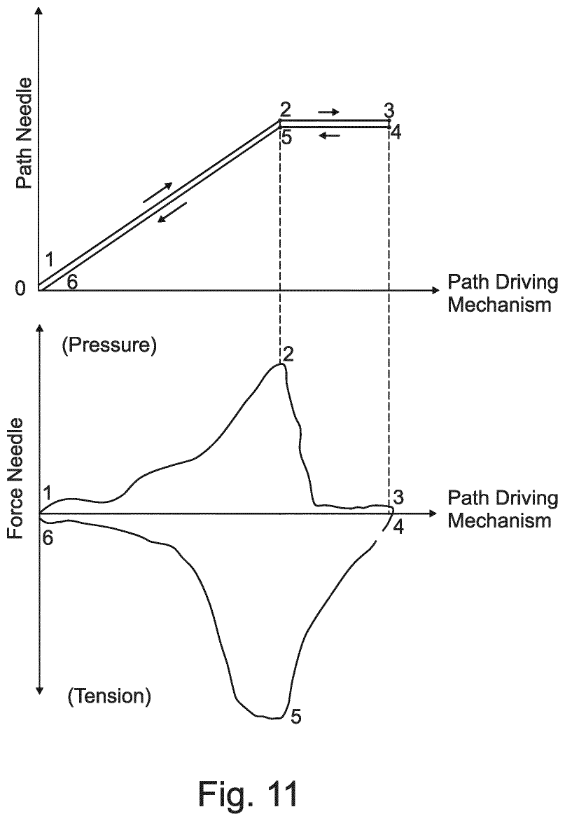

FIG. 11 shows, in a top chart, a displacement of the felting needle, in particular of the working tip 7 of the felting needle 6, as a function of the longitudinal displacement of the driving mechanism. At a point 1, the working tip 7 is fully retracted. The path between points 1 and 2 is indicative of the working tip 7 being extended. At a point 2, the working tip 7 contacts a hard object which it cannot penetrate and therefore the extension of the working tip 7 is arrested. On the path between points 2 and 3, the working tip 7 does not further extend, even though the driving mechanism 14 continues to extend in the longitudinal direction. This is due to the force exerted by the driving mechanism 14 on the working tip exceeding the predetermined force threshold and, as a consequence, the force limiting element 15 engaging. At point 3, the driving mechanism 14 has reached its maximum longitudinal extension. At point 4, the driving mechanism 15 begins its return path. Between points 4 and 5, the driving mechanism 15 retracts, however the working tip 7 remains stationary. At point 5, the working tip 5 loses contact with the hard object and follows the displacement of the driving mechanism 14 back to the end point 6. The forward and return paths do not necessarily overlap due to a hysteresis, for example in the force limiting element 15.

In a bottom chart the force on the working tip 7 of the needle 6 is illustrated as the needle follows the path shown in the top chart. Between points 1 and 2, the working tip 7 moves through soft tissue, as shown by the irregular force exerted on the working tip. At point 2, the working tip 7 encounters a hard object, such as hard tissue (e.g., bone), or another surgical instrument (e.g., a scalpel). The force exerted on the working tip 7 meets the predetermined force threshold and the force limiting element 15 engages, thereby preventing the working tip 7 from exerting a force on the hard object in excess of the predetermined force threshold. Further displacement by the driving mechanism 14 from point 2 to 3 is met with a sharp decrease in the force exerted by the working tip 7 on the hard object, as shown in the trajectory of the force curve between points 2 and 3. The force may suddenly decrease to a minimal force level, which minimal force level may be near zero. On the return path, i.e. from points 4 to 5, the force exhibited on the working tip 7 may follow a different path than the forward path (i.e. path from point 1 to point 3) due to hysteresis. For example, the force on the working tip 7 may become negative (i.e. directed in a dorsal direction) at least for a part of the return path. In particular, the force may become negative between points 5 and 6, during which the working tip 7, which may have become partially embedded in the hard object, is pulled from the hard object and then is pulled back out through the soft tissue. In particular, the force limiting element 16 may not engage on the return path, such that the magnitude of the force on the path between points 4 and 6 may exceed the predetermined force threshold. Thereby, even if the working tip 7 becomes embedded or snagged it can be removed by the driving mechanism

LIST OF DESIGNATIONS

| | |
|---|---|
| 1 | Surgical device |
| 4 | Carrier |
| 5 | Guide rod (carrier) |
| 6 | Felting needle (carrier) |
| 7 | Working tip (needle) |
| 8 | Guide bushing |
| 9 | Extended position |
| 10 | Retracted position |
| 11 | Aperture (guide bushing) |
| 12 | Distal end (guide rod) |
| 13 | Dorsal end (guide rod) |
| 14 | Driving mechanism |
| 15 | Force limiting element |
| 16 | Buckling element/Buckling bar |
| 17 | First end (buckling element) |
| 18 | Second end (buckling element) |
| 19 | Distal end (guide bushing) |
| 20 | Dorsal end (guide bushing) |
| 21 | Frame (surgical device) |
| 22 | Recess (frame) |
| 23 | Channel |
| 24 | Adapter (driving mechanism) |
| 25 | Actuator |
| 26 | Guide bushing collar |
| 27 | Piston rod |
| 28 | Outer housing |
| 29 | Indentation (guide rod) |
| 30 | Guide surface (guide rod) |
| 31 | Biasing member |
| 32 | Bore |
| 40 | Outer shaft (working tip) |
| 41 | Flat end section |
| 42 | Bevels |

1. A surgical device for attaching a non-woven textile to human or animal soft tissue by releasing sections of individual fibers of the non-woven textile and carrying them into the human or animal soft tissue, the device comprising:
a. a carrier extending in a longitudinal direction, comprising a guide rod and a felting needle attached to a distal end of the guide rod, wherein
b. the guide rod and the felting needle are at least partially arranged in a guide bushing and the felting needle is reciprocally movable between
i. an extended position in which a working tip of the felting needle protrudes beyond an aperture arranged at a distal end of the guide bushing and
ii. a retracted position in which the working tip of the felting needle does not protrude beyond the aperture arranged at the distal end of the guide bushing; wherein
c. the guide rod comprises a dorsal end which is coupled to a driving mechanism via a force limiting element which during operation limits the force applied by the driving mechanism to the guide rod to a predetermined force threshold when the working tip is moved from the retracted position to the extended position.

2. The surgical device according to claim 1, wherein the force limiting element is further configured to not limit the force applied by the driving mechanism to the guide rod to the predetermined force threshold when the working tip is moved from the extended position to the retracted position.

3. The surgical device according to claim 1, wherein the force limiting element comprises a deformable element which deforms when the predetermined force threshold is reached in longitudinal direction thereby interrupting the force transmission in longitudinal direction.

4. The surgical device according to claim 3, wherein the deformable element is a buckling element which extends between a first end and a second end and deforms in a lateral direction when the predetermined force threshold is reached.

5. The surgical device according to claim 4, wherein the buckling element is clamped at the first end and/or the second end.

6. The surgical device according to claim 3, wherein the buckling element is preformed such that the buckling element buckles when the pre-determined force threshold is reached during operation.

7. The surgical device according to claim 1, wherein the working tip of the felting needle includes carrying means for supporting the carrying of sections of the individual fibers into the soft tissue.

8. The surgical device according to claim 7, wherein the carrying means include an essentially flat end section arranged at the distal end of the working tip and/or barbs along an outer shaft of the working tip.

9. The surgical device according to claim 1, wherein the surgical device comprises a frame to which the guide bushing and the driving mechanism are attached.

10. The surgical device according to claim 9, wherein the frame comprises a recess in which the force limiting element is arranged between the dorsal end of the guide bushing and the driving mechanism.

11. The surgical device according to claim 1, wherein a channel extends in longitudinal direction between the guide rod and the guide bushing.

12. The surgical device according to claim 11, wherein the guide rod comprises a channel which extends in longitudinal direction.

13. The surgical device according to claim 1, wherein the driving mechanism comprises an adapter to connect the driving mechanism to an external actuator.

14. The surgical device according to claim 1, wherein the aperture is formed by guide bushing collar which has an inner diameter less than an inner diameter of the guide bushing, wherein the guide bushing collar includes a transitional surface at a distal end and/or at a dorsal end.

15. The surgical device according to claim 1, wherein the felting needle is made from a metal and the guide rod is made from an injection molded plastic material encompassing the felting needle opposite to the working tip at least partially.

16. The surgical device according to claim 15, wherein the force limiting element and the felting needle are integrally formed and extend through the guide rod.

17. The surgical device according to claim 1, wherein the guide rod comprises at least two opposite indentations between which a part of the force limiting member is arranged.

18. The surgical device according to claim 1, wherein the force limiting element is coupled to the driving mechanism by a piston rod.

19. The surgical device according to claim 1, wherein the piston rod is linearly guided in a bore of the frame.

20. The surgical device according to claim 1, wherein the frame is at least partially encompassed by an outer housing or, wherein the guide rod comprises at least two guide surfaces spaced apart from each other in longitudinal direction.

21. (canceled)
